(19) United States
(12) Patent Application Publication
(10) Pub. No.: US 2008/0108827 A1
(43) Pub. Date: May 8, 2008

(54) PROCESS FOR PREPARING RILUZOLE

(76) Inventors: Pratap Reddy Padi, Hyderabad (IN); Madhusudhan Reddy Ganta, Hyderabad (IN); Satyanarayana Bollikonda, Hyderabad (IN); Sridhar Reddy Chaganti, Hyderabad (IN); Ramulu Akula, Warangal (IN); Loka Maheshwari Pochaiah Dommati, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/923,804

(22) Filed: Oct. 25, 2007

Related U.S. Application Data
(60) Provisional application No. 60/885,477, filed on Jan. 18, 2007.

(30) Foreign Application Priority Data
Oct. 25, 2006 (IN) 1958/CHE/2006

(51) Int. Cl. C07D 279/08

(52) U.S. Cl. 548/164

(57) ABSTRACT

The present application provides a process for the preparation of riluzole, which process includes reacting 4-trifluoromethoxyaniline of Formula II with ammonium thiocyanate of Formula III.

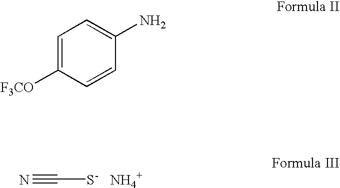

### INTRODUCTION TO THE INVENTION

The present invention relates to a process for the preparation of riluzole.

Riluzole is chemically described as 2-amino-6-(trifluoromethoxy)benzothiazole (hereinafter referred by its adopted name “riluzole”) and can be represented by Formula I.

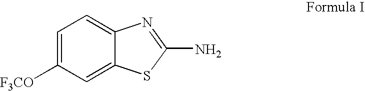

Riluzole is a glutamate antagonist, is a member of the benzothiazole class and is useful in the treatment of patients with amyotrophic lateral sclerosis. It is sold in the market under the brand name RILUTEK™ as tablets in the dosage strengths of 10 mg, 25 mg and 50 mg.

U.S. Pat. No. 4,370,338 describes riluzole as a useful anticonvulsant, anxiolytic, and hypnotic drug.

The patent also discloses a process for the preparation of riluzole which involves adding bromine in acetic acid dropwise to a solution of 4-trifluoromethoxyaniline and potassium thiocyanate in acetic acid. The reaction mixture is stirred overnight at ambient temperature and is then poured into 2 liters of water, cooled in an ice bath, and neutralized with ammonia. The resultant insoluble 2-amino-6-trifluoromethoxy-benzothiazole (riluzole) is collected by filtration and recrystallized from a 50:50 ethanol-water mixture.

Jimonet et al, Journal of Medicinal Chemistry, 1999, vol. 42, No. 15, pages 2828-2843 describe the synthesis of homologues of riluzole using ammonium thiocyate by a two-step process.

The present invention provides a process, which is simple, efficient, inexpensive, ecofriendly, robust, and readily scaleable.

### SUMMARY OF THE INVENTION

The present invention provides a process for the synthesis of riluzole of Formula I.

In one aspect, the present invention relates to a process for the preparation of riluzole of Formula I. The process comprises: reaction of the compound 4-trifluoromethoxyaniline of Formula III with the compound ammonium thiocyanate of Formula II in the presence of suitable organic solvent(s) followed by cyclization of the corresponding intermediate compound using suitable cyclizing agent to afford the desired compound 2-amino-6-(trifluoromethoxy)benzothiazole of Formula I.

Riluzole of Formula I obtained by the above process is characterized by the X-ray powder diffraction having principal peaks at approximately: 9.1, 13.6, 18.1, 21.1, 22.7, 25.2, and 34.1, ±0.2 degrees 2 theta, which is substantially in accordance with FIG. 2.

In another aspect, the present invention relates to pharmaceutical compositions comprising riluzole of Formula I made by the process of the invention and at least one pharmaceutically acceptable carrier.

### BRIEF DESCRIPTION OF THE DRAWINGS

FIG. 1: is a schematic representation of a process for preparation of the compound of Formula I.

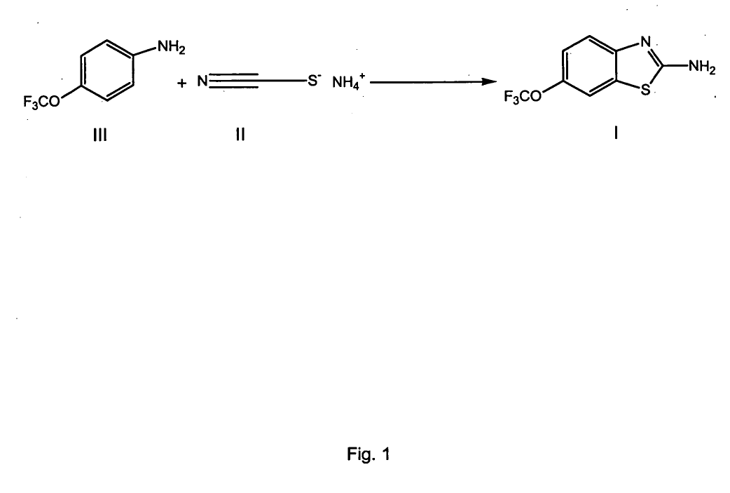

FIG. 2: is a characteristic powder X-ray diffraction pattern of the riluzole compound of Formula I.

### DETAILED DESCRIPTION OF THE INVENTION

The present invention provides process for the synthesis of riluzole of Formula I,

In one embodiment of the present invention, there is provided a process for the preparation of riluzole of Formula I. The process comprises: reaction of the compound 4-trifluoromethoxyaniline of Formula III

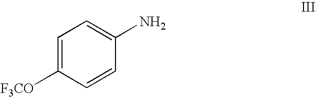

with the compound ammonium thiocyanate of Formula II

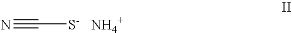

in the presence of suitable organic solvent(s) followed by cyclization of the corresponding intermediate compound using suitable cyclizing reagent to afford the desired compound 2-amino-6-(trifluoromethoxy)benzothiazole of Formula I.

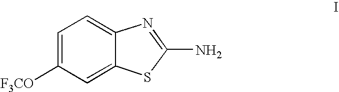

Suitable organic solvents include but are not limited to acidic solvents such as acetic acid, hydrochloric acid, sulfuric acid and the like

Suitable cyclizing agents include but are not limited to bromine in acetic acid, sulfuric acid, formic acid anhydrous bromine gas and the like or mixtures thereof.

Suitable temperatures for conducting the reaction include about 25° C. to 75° C., or about 60° C. to 65° C., or about the reflux temperature of the solvent used.

The reaction can be conducted as long as required for the completion of the reaction, from about 30 minutes to about 10 hours frequently being required.

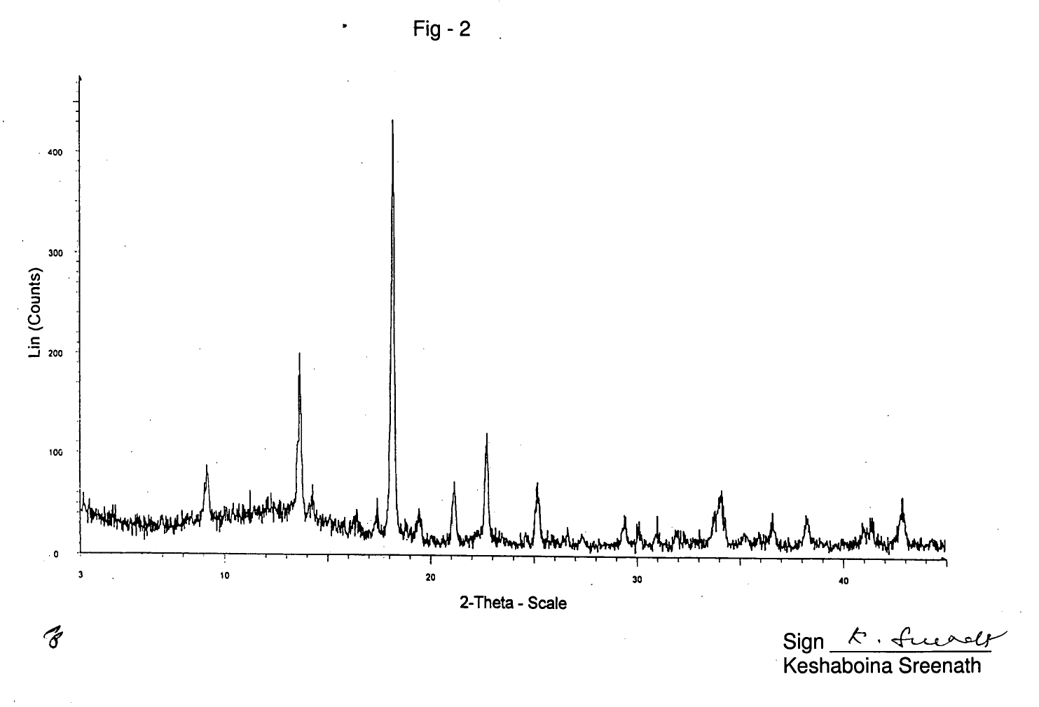

Riluzole of Formula I obtained by the above processes is characterized by the X-ray powder diffraction having principal peaks at approximately: 9.1, 13.6, 18.1, 21.1, 22.7, 25.2, and 34.1, ±0.2 degrees 2 theta. which is substantially in accordance with FIG. 2. The pattern was measured on a Bruker Axe, D8 Advance Powder X-ray Diffractometer with a Cu K alpha-1 radiation source.

The solid riluzole of Formula I thus obtained is recovered from the reaction mixture by suitable techniques such as decantation, filtration by gravity or by suction, centrifugation, and the like.

The wet cake is optionally subjected to further drying. Drying can be suitably carried out in a tray dryer, vacuum oven, air oven, fluidized bed drier, spin flash dryer, flash dryer and the like.

The drying can be carried out at temperatures of about 25° C. to about 75° C. for any desired time period to achieve a desired result, time cycles from about 1 to 20 hours frequently being suitable.

The residue or solid product of Formula I thus obtained may be optionally purified by recrystallization using suitable organic solvents or their mixtures or their aqueous mixtures thereof.

Typically, riluzole of Formula I is dissolved in a minimal amount of the solvent system, thereafter the solution is cooled slowly until to afford the desired pure form of riluzole of Formula I.

Recrystallising the product riluzole of step (b) using suitable solvents at suitable temperature(s) for suitable time provides the pure form of riluzole of Formula I.

Suitable organic solvents include but are not limited to: alcohols such as methanol, ethanol, n-propanol, isopropyl alcohol, n-butanol, tertiary-butyl alcohol and the like; ketones such as acetone, methyl ethyl ketone, methyl isobutyl ketone, and the like; hydrocarbon solvents such as toluene, xylene, cyclohexane and the like; esters such as ethyl acetate, isopropyl acetate, tertiary butyl acetate and the like; nitrites such as acetonitrile, propionitrile and the like; and mixtures thereof or their combinations with water in various proportions.

Suitable temperatures for recrystallisation include about 0° C. to about 75° C., or about 50° C. to about 60° C., or about reflux temperature of the solvent used. The process can be conducted from about 10 minutes to about 5 hours, or longer.

The concentration of the riluzole in the solvent can range from 40 to 80% or more. The solution can be prepared at an elevated temperature if desired to achieve a higher solute concentration. Any temperature is acceptable for the dissolution as long as a clear solution of the riluzole is obtained and is not detrimental to the drug substance chemically or physically. The solution may be brought down to a lower temperature for further processing if required or an elevated temperature may be used. A higher temperature for dissolution will allow the precipitation from solutions with higher concentrations of riluzole, resulting in better economies of manufacture.

The product may optionally be further dried. Drying can be suitably carried out in a tray dryer, vacuum oven, air oven, fluidized bed drier, spin flash-dryer, flash dryer and the like.

The drying can be carried out at temperatures of about 35° C. to about 70° C. The drying can be carried out for any desired time periods to achieve the desired product purity, times from about 1 to 20 hours frequently being adequate.

The compound of Formula I may be prepared in crystalline or non-crystalline form, and if crystalline, may optionally be solvated, e.g., as the hydrate. The process according to the present invention can provide substantially pure riluzole of Formula I.

The process according to the present invention preferably yields substantially pure riluzole, which has a purity greater than or equal to about 99.9 wt. %. Correspondingly, the total impurities will be not more than about 0.1 wt. % and any single maximum impurity is less than or equal to about 0.01 wt. % by high performance liquid chromatography (HPLC).

The riluzole of Formula I is substantially free from impurities both process and structural impurities. Typically the riluzole is of high purity such as at least about 99.5 wt %, or at least about 99.9 wt % pure. Correspondingly, the level of impurities may be less than about 0.5 wt %, 0.1 wt %, or 0.01 wt % by high performance liquid chromatography (HPLC).

Similarly, the riluzole of Formula I is substantially free from residual solvents such as solvents used in making the riluzole. The residual solvent content may be less than about 10 wt %, or less than about 2 wt %, or less than about 1 wt %, 0.5 wt %, or 0.1 wt %.

In yet another aspect, the present invention encompasses pharmaceutical compositions comprising riluzole of Formula I made by the process of the present invention and at least one pharmaceutically acceptable carrier.

The drug substance can be formulated as solid compositions for oral administration, tablets, pills, powders or granules may be used. In these compositions, the active product according to the invention is mixed with one or more inert diluents such as sucrose, lactose or starch. These compositions can also comprise substances other than diluents, e.g. a lubricant such as magnesium stearate.

The drug substance can be formulated as liquid compositions for oral administration, solutions, suspensions, syrups, elixirs and pharmaceutically acceptable emulsions, containing inert diluents such as water or liquid paraffin, may be used. These compositions can also comprise substances other than diluents, e.g. wetting, sweetening or flavouring products.

The compositions for parenteral administration can be suspensions, emulsions or aqueous or non-aqueous, sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable carriers that are of use in the present invention are but not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, polyvinyl pyrrolidone, hydroxypropyl cellulose, hydroxypropyl rriethylcellulose, pregelatinized starch and the like; disintegrants such as starch, sodium starch glycolate, pregelatinized starch, crospovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, complex forming agents such as various grades of cyclodextrins, resins; release rate controlling agents such as hydroxypropyl cellulose, hydroxymethyl cellulose, hydroxypropyl methyl cellulose, ethyl cellulose, methyl cellulose, various grades of methyl methacrylates, waxes and the like. Other pharmaceutically acceptable excipients that are of use include but not limited to film formers, plasticizers, colorants, flavoring agents, sweeteners, viscosity enhancers, preservatives, antioxidants and the like.

The process of the present invention is efficient, cost effective, ecofriendly, reproducible, scalable, robust and commercially feasible.

The process of the present invention produces the desired compound riluzole of Formula I with high yield and purity.

Certain specific aspects and embodiments of the invention are further described in the example given below, which is provided by way of illustration only and therefore should not be construed to limit the scope of the invention.

### EXAMPLE 1

### Process for the Preparation of 2-Amino-6-(Trifluoromethoxy) Benzothiazole (Formula I)

115 ml of acetic acid, 25 g of 4-trifluoromethoxyaniline of Formula III and 26.25 g of ammonium thiocyanate of Formula II were charged in a clean and dry 4 neck round bottom flask followed by cooling to about 10° C. A mixture of 7.3 ml of bromine and 50 ml of acetic acid was added over about 1 hour, 20 minutes. The resultant reaction mixture was allowed to reach the temperature of about 27° C. followed by stirring for about 5.5 hours. After completion of the reaction, the reaction mixture was quenched by adding the reaction mixture to 150 ml of precooled water. The undissolved solids were filtered and the resultant filtrate was washed with 2×100 ml of toluene. The reaction solution was cooled to about 5° C. followed by adjusting the pH to 7.5 by the addition of 290 ml of ammonium hydroxide. The resultant reaction suspension was stirred at about 9° C. for about 45 minutes. The separated solid was filtered and the solid was washed with 150 ml of water. 100 ml of methanol, 50 ml of water and the solid obtained were charged in a clean and dry 4 round bottom flask followed by heating to about 53° C. 2 g of charcoal carbon was charged followed by stirring for about 25 minutes. The reaction suspension was filtered through a celite bed and the celite was washed with 25 ml of methanol. The resultant filtrate was charged in a clean and dry 4 neck round bottom flask followed by charging of 75 ml of water. The solution was cooled to about 5° C. followed by stirring for about 1 hour. The solid separated and was filtered and the solid was washed with 1:3 ratio of methanol and water mixture (100 ml). The solid obtained was dried at about 66° C. for about 5 hours to afford 24.2 g of the title compound with purity by HPLC of 99.95%.

1. A process for preparing riluzole of the Formula I: 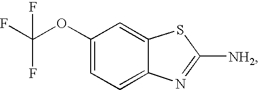
which process comprises reacting 4-trifluoromethoxyaniline of Formula II: 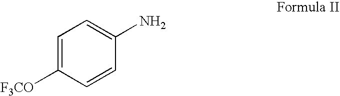
with ammonium thiocyanate of Formula III:

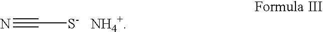

2. The process of claim 1, wherein said reaction is carried out at a temperature ranging from about 20° C. to about 35° C.
